(19) United States
(12) Patent Application Publication
(10) Pub. No.: US 2007/0122360 A1
(43) Pub. Date: May 31, 2007

(54) Nonaqueous gel composition for tooth whitening and tooth whitening set

(75) Inventors: Takayuki Oniki, Sumida-ku (JP); Akira Uchiyama, Sumida-ku (JP); Yasushi Fukuda, Sumida-ku (JP); Shimako Inoue, Sumida-ku (JP)

Correspondence Address: BIRCH STEWART KOLASCH & BIRCH, PO BOX 747, FALLS CHURCH, VA, 22040-0747, US

(73) Assignee: LION CORPORATION, TOKYO (JP)

(21) Appl. No.: 10/584,192

(22) PCT Filed: Dec. 24, 2004
(86) PCT No.: PCT/JP04/19344

§ 371 Date: Jun. 23, 2006

(30) Foreign Application Priority Data
Dec. 26, 2003 (JP) 2003-432095

(51) Int. Cl. A61K 8/81

(52) U.S. Cl. 424/53

(57) ABSTRACT

A nonaqueous gel composition for tooth whitening, comprising: (A) tooth whitening ingredient having a relative permittivity of 17.0 to 43.0 (at 25° C.) and a vapor pressure of 0 to 7000 kPa (at 25° C.), (B) substance that is soluble in the tooth whitening ingredient and is precipitated by an aqueous solution of calcium chloride, and (C) gelling agent, which nonaqueous gel composition contains substantially none of water and peroxide. This tooth whitening component can penetrate into the enamel surface layer of teeth to thereby alter the optical properties, such as refractive index and reflectance, of the enamel so that the enamel is apparently whitened. The whitened teeth can be restored to the original tooth color in a reversible manner in the presence of water. Further the persistence of reversible whitening effect can be strikingly enhanced by the nonaqueous gel composition.

### TECHNICAL FIELD

The present invention relates to a composition which, upon application to teeth, makes teeth look whiter than their original color and keeps long its whitening effect. More particularly, the present invention relates to a nonaqueous gel composition for tooth whitening and a tooth whitening set, the former containing a tooth whitening ingredient that penetrates into the enamel of the tooth for replacement of water therein, thereby changing the optical properties (such as refractive index and reflectivity) of the enamel without chemical reactions (such as bleaching) so that the treated teeth apparently look white and this whitening effect remains long.

### BACKGROUND ART

Among conventional technologies of making teeth look white are (1) the one which involves removal of stains from teeth for restoration of the original tooth whiteness and (2) the one which is intended to make teeth look whiter than original. The first includes physical procedure such as brushing with a tooth brush and a dentifrice containing an abrasive and chemical procedure such as decomposition of stains by a solubilizer (e.g., polyethylene glycol and polyvinyl pyrrolidone), enzyme, chelating agent, or the like. The second includes bleaching with a peroxide (common practice in Europe), coating with a masking agent (typified by tooth manicure), and laminate veneer technique.

The recent increasing interest in dental health and aesthetic appreciation has shifted the procedure for tooth whitening from “cleaning” to “making teeth look white”. However, bleaching with a peroxide (which might cause gingival inflammation and retraction) is dangerous for consumers without expert knowledge. On the other hand, coating with manicure lacks natural appearance and durability (it easily peels off after eating and drinking). The laminate veneer technique belongs to the dentist's domain. Moreover, it needs the grinding of the surface of healthy teeth, and the ground teeth cannot be restored even though the patient is not satisfied with the results of operation.

For the reasons mentioned above, there has been a demand for development of a new technology for tooth whitening, which meets requirements for aesthetical appreciation, simple and safe use (without peroxide), and easy restoration of original colors.

Among conventional reversible whitening techniques is manicure coating with a film-forming material such as shellac, vinyl acetate resin, and acrylic resin (Japanese Patent Laid-open Nos. Hei 04-82821, Hei 05-58844, Hei 09-100215, Hei 09-202718, and Hei 09-151123). However, these prior art technologies still have room for improvement in color tone and durability.

The present inventors previously proposed a whitening method not involving chemical reactions such as bleaching. This method involves the modification of enamel in optical properties which makes teeth apparently look white (PCT Patent Publication No. WO03/030851). After several minutes of treatment according to this method, the patient really feels his or her teeth having become white. Unfortunately, this whitening effect does not last long.

Patent Document 1:
Japanese Patent Laid-open No. Hei 04-82821

Patent Document 2:
Japanese Patent Laid-open No. Hei 05-58844

Patent Document 3:
Japanese Patent Laid-open No. Hei-09-100215

Patent Document 4:
Japanese Patent Laid-open No. Hei 09-202718

Patent Document 5:
Japanese Patent Laid-open No. Hei 09-151123

Patent Document 6:
PCT Patent Publication No. WO03/030851

### DISCLOSURE OF THE INVENTION

### Problems to be Solved by the Invention

It is an object of the present invention to provide a nonaqueous gel composition for tooth whitening and a tooth whitening set, which change the optical properties of the enamel without using any peroxide, thereby making teeth apparently look white while permitting teeth to restore their original color in the presence of water and which keep the whitening effect over a long period of time.

### Means for Solving the Problems

The present inventors carried out extensive investigations on the method for whitening teeth with a composition containing a tooth whitening ingredient which penetrates into the enamel of the tooth for replacement of water therein, thereby changing the optical properties (such as refractive index and reflectivity) of the enamel without chemical reactions (such as bleaching) so that the treated teeth apparently look white. As the result, they found that it is possible to protect the tooth whitening ingredient, which has once penetrated into the enamel, from leaching out of the enamel by incorporation with a substance (such as C₁₄₋₂₂ higher fatty acid and acrylic acid copolymer) which dissolves in the tooth whitening ingredient and is precipitated by an aqueous solution of calcium chloride. The substance will be referred to as a whitening effect endurance improver or an ingredient (B) hereinafter. The foregoing finding led to the present invention.

The present invention is directed to a nonaqueous gel composition for tooth whitening which includes

(A) a tooth whitening ingredient having a relative permittivity of 17.0 to 43.0 (at 25° C.) and a vapor pressure of 0 to 7000 kPa (at 25° C.),

(B) a substance which dissolves in the tooth whitening ingredient and is precipitated by an aqueous solution of calcium chloride, and

(C) a gelling agent,

the composition being substantially free of water and peroxide.

The tooth whitening ingredient (A) mentioned above should preferably be any one or more compounds selected from among isopropanol, butanol, ethylene glycol, polyethylene glycol (with an average molecular weight of 190 to 630), diethylene glycol, propylene glycol, dipropylene glycol, butylene glycol, and glycerin, and the substance (B) mentioned above should preferably be any one or more compounds selected from among C₁₄₋₂₂ higher fatty acids and acrylic acid copolymers.

The tooth whitening composition according to the present invention achieves its object in such a way that the tooth whitening ingredient penetrates into the enamel of the tooth and stays there, thereby changing the enamel in optical properties (such as refractive index and reflectance) without requiring any peroxide and making the tooth apparently look whiter than its original color. In addition, it keeps long the tooth whitening effect and yet it permits the treated tooth to restore its original color in the presence of water.

The present invention is directed also to a tooth whitening set which includes the nonaqueous gel composition for tooth whitening defined above and a tool for its application which is detachably fitted to teeth while holding it. The tool for application may be any of tape, sheet, film, mouth tray, mouth piece, sponge, impression material, pack material, and tooth cover conforming to the dentition, which are all made of water-insoluble material.

The tool for application should be useful for the tooth whitening method.

### ADVANTAGEOUS EFFECTS OF THE INVENTION

The present invention produces the effect of making the enamel of the tooth apparently look white while allowing the restoration of the original tooth color in the presence of water and keeping long the reversible whitening effect owing to the tooth whitening ingredient which penetrates into the enamel layer of the tooth, thereby changing the enamel in its optical properties such as refractive index and reflectance.

### BEST MODE FOR CARRYING OUT THE INVENTION

The nonaqueous gel composition for tooth whitening according to the present invention contains the ingredient (A) as a tooth whitening ingredient which has a relative permittivity of 17.0 to 43.0, preferably 30.0 to 43.0 (at 25° C.) and a vapor pressure of 0 to 7000 kPa, preferably 0 to 700 kPa (at 25° C.). With a relative permittivity smaller than 17.0, the tooth whitening ingredient is too poor in water miscibility and ability to penetrate into the enamel to fully produce the whitening effect. With a relative permittivity larger than 43.0, the tooth whitening ingredient will not appreciably change the enamel in optical properties even though it has replaced water in the enamel. With a vapor pressure higher than 7000 kPa, the tooth whitening ingredient will vaporize at the time of application to the enamel without fully producing the whitening effect. The lower limit of the vapor pressure is not specified although it is usually higher than 0 kPa.

Examples of the tooth whitening ingredient include isopropanol, 1,2-propylene glycol, 1,3-propylene glycol, ethylene glycol, dipropylene glycol, diethylene glycol, polyethylene glycol (with an average molecular weight of 190 to 630), 1,3-butylene glycol, butanol, and glycerin. Among preferred examples are 1,2-propylene glycol, 1,3-propylene glycol, diethylene glycol, polyethylene glycol (with an average molecular weight of 190 to 630), 1,3-butylene glycol, glycerin, and ethylene glycol. Most desirable examples are polyethylene glycol (with an average molecular weight of 190 to 630), 1,3-butylene glycol, and glycerin.

Incidentally, the average molecular weight is the one which is defined in the Japanese standards of cosmetics ingredient (annotation to the 2nd edition). “Polyethylene glycol (with an average molecular weight of 190 to 630)” includes polyethylene glycol 200 (with an average molecular weight of 190 to 210), polyethylene glycol 300 (with an average molecular weight of 280 to 320), polyethylene glycol 400 (with an average molecular weight of 380 to 420), and polyethylene glycol 600 (with an average molecular weight of 570 to 630). The number following “polyethylene glycol” may be preceded by # (like #200).

The nonaqueous gel composition for tooth whitening according to the present invention may be incorporated with 0 to 20 wt % of liquid ingredient (such as ethanol), in addition to the tooth whitening ingredient, for improvement in handleability at the time of application to teeth. The ratio of the tooth whitening ingredient to the liquid ingredient (such as ethanol) should be from 60/40 to 100/0 (by weight), preferably from 75/25 to 100/0 (by weight). If the ratio is smaller than 60/40, the tooth whitening ingredient will not fully produce its effect.

The nonaqueous gel composition for tooth whitening according to the present invention contains the tooth whitening ingredient which accounts for 0.0 to 99.5 wt %, preferably 60.0 to 99.0 wt %, in the entire composition. The tooth whitening ingredient is not specifically restricted in its amount so long as it dissolves out of the tooth whitening nonaqueous gel composition.

The nonaqueous gel composition for tooth whitening according to the present invention contains the ingredient (B) which dissolves in the tooth whitening ingredient and is precipitated by an aqueous solution of calcium chloride. The ingredient (B) is a whitening effect endurance improver.

The term “dissolves in the tooth whitening ingredient” means that the ingredient (B) has a solubility higher than 0.1 g in 100 g of the tooth whitening ingredient at 37° C. The term “is precipitated by an aqueous solution of calcium chloride” means that the ingredient (B) has a solubility lower than 0.1 g in 100 g of aqueous solution of 1 mmol/L calcium chloride at 37° C. The ingredient (B) may be solid or liquid at normal temperature. If the ingredient (B) is liquid, the term “is precipitated by an aqueous solution of calcium chloride” means that the ingredient (B) forms precipitates or becomes turbid. The aqueous solution of calcium chloride used to evaluate the solubility of the ingredient (B) has a concentration of 1 mmol/L which is close to the calcium ion concentration in saliva.

The solubility of the ingredient (B) is evaluated by visually observing residues that remain undissolved when 0.1 g of the ingredient (B) is added to 100 g of the tooth whitening ingredient (A) and the resulting mixture is stirred at 37° C. for 16 hours and then allowed to stand at 37° C. for 24 hours. The same procedure as mentioned above is also employed to evaluate the solubility of 0.1 g of the ingredient (B) in 100 g of aqueous solution of 1 mmol/L calcium chloride.

According to the present invention, the ingredient (B) prevents the tooth whitening ingredient, which has penetrated into the enamel of the tooth, from leaching out of the enamel, thereby allowing the tooth whitening effect to last longer. In addition, the ingredient (B) is precipitated by saliva on the gel surface not in contact with the tooth being treated, thereby preventing the nonaqueous gel composition for tooth whitening from dissolving in the mouth.

The ingredient (B) mentioned above should preferably be a C₁₄₋₂₂ higher fatty acid and/or acrylic acid copolymer. The higher fatty acid should preferably be that of linear, branched, or cyclic structure, with 14 to 22 carbon atoms, which dissolves in the tooth whitening ingredient and is precipitated by an aqueous solution of calcium chloride. It may include hydroxy higher fatty acids which have hydroxyl groups attached to arbitrary carbons. Two or more species of the higher fatty acid may be connected to each other through an ester linkage or lactone linkage. The acrylic acid copolymer also includes methacrylic acid copolymer. The acrylic acid copolymer may have the carboxyl group in the form of acid, alkyl ester, ammonium alkyl ester, or amide. The ingredient (B) may be composed of two or more whitening effect endurance improvers.

The higher fatty acid used in the present invention should be one which dissolves in the tooth whitening ingredient and is precipitated by an aqueous solution of calcium chloride. It includes those of linear, branched, and cyclic structure. It should have a carbon number or 14 to 22, preferably 15 to 18. It is not specifically restricted in the number and type of unsaturated bonds and in the structure of geometric isomer. It should preferably be one which has 0 to 3 double bonds. It is not specifically restricted in the number of branched chains. The one with a branched carbon is preferable. Preferred higher fatty acids of cyclic structure include those of tricyclic structure, particularly those represented by the structural formula (1) below. The higher fatty acid includes hydroxy higher fatty acid, which is not specifically restricted in the position and number of hydroxyl groups. The number of hydroxyl groups should preferably be 0 to 3.

[Chemical Formula 1]

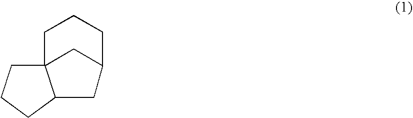

The higher fatty acids may be used in combination in the present invention. The compound having ester linkage or lactone linkage between two or more higher fatty acids is also preferably used. The bonding ratio in case of bonding the tricyclic structure-containing higher fatty acid and the other higher fatty acid is not restricted, although the weight ratio of the other fatty acid/the tricyclic structure-containing fatty acid should preferably be from 10/90 to 90/10, more preferably 30/70 to 70/30. Such a higher fatty acid is exemplified by shellac.

Shellac is a natural resin secreted by the insect Laccifer lacca according to the catalog of Japan Shellac Industries, Ltd. and “Kagaku to Kogyo” vol. 51, pp. 356 to 361. It is a condensation product formed from hydroxyl carboxylic acids through ester linkage or lactone linkage. Analytical study by hydrolysis suggests that shellac resin is composed of 9,10,16-trihydroxypalmitic (ca. 40%) and jalaric acid and derivatives thereof (ca. 40%).

Examples of the higher fatty acids include myristic acid, 7-hydroxymyristic acid, jalaric acid, 9,10,16-trihydroxypalmitic acid, palmitoleic acid, 12-hydroxystearic acid, isostearic acid, oleic acid, linoleic acid, linolenic acid, erucic acid, and shellac. Preferable among these examples are jalaric acid, 9,10,16-trihydroxypalmitic acid, palmitoleic acid, 12-hydroxystearic acid, isostearic acid, oleic acid, linoleic acid, linolenic acid, and shellac. Jalaric acid, isostearic acid, shellac are particularly preferable.

The acrylic acid copolymer used in the present invention is one which is formed from at least two monomer selected from the group consisting of acids, amides, alkyl esters, and ammonium alkyl esters of acrylic acid and methacrylic acid. The alkyl group should preferably be one which has 1 to 4 carbon atoms. Examples of the acrylic acid copolymer include

t-Bu acrylate/ethyl acrylate/methacrylic acid copolymer, methyl acrylate/methacrylic acid copolymer, methyl acrylate/methacrylic acid copolymer, methyl methacrylate/methacrylic acid copolymer, acrylic acid/acrylamide/ethyl acrylate copolymer, methyl methacrylate/ethyl acrylate/methacrylic acid trimethylammonium ethyl chloride copolymer, and octylacrylamide/acrylate ester copolymer.

Preferable among these examples are

t-Bu acrylate/ethyl acrylate/methacrylic acid copolymer, methyl methacrylate/ethyl acrylate/methacrylic acid trimethylammonium ethyl chloride copolymer, acrylic acid/acrylamide/ethyl acrylate copolymer, and octylacrylamide/acrylate ester copolymer. They are not specifically restricted in weight-average molecular weight (determined by gel filtration method using THF as a solvent and polystyrene as a standard substance), with a desirable value being 50,000 to 2,000,000, particularly 100,000 to 1,000,000.

The nonaqueous gel composition for tooth whitening according to the present invention is based on the principle that, upon application to teeth, it remains on the surface of teeth and its tooth whitening ingredient penetrates into the enamel for replacement of water in the crystals of the enamel, thereby changing the enamel in optical properties (refractive index and reflectance) and causing the enamel to apparently look turbid. Involving no chemical reactions, it is highly safe. Several hours after application, the tooth whitening ingredient which has penetrated into the enamel is gradually replaced by water in saliva, so that the treated teeth restore their original color. In other words, the tooth whitening is achieved in a reversible manner. However, the tooth whitening ingredient contains a higher fatty acid or acrylic acid copolymer (as the whitening effect endurance improver) dissolved therein, which is precipitated by saliva, thereby preventing the tooth whitening ingredient from leaching out and also preventing the treated teeth from restoring their original color.

The whitening effect endurance improver used in the present invention is not specifically restricted in amount. A preferred amount is 0.1 to 10 wt %, particularly 0.1 to 5 wt %, of the total amount of the composition. With an amount less than 0.1 wt %, it does not entirely prevent the tooth whitening ingredient from leaching out of the enamel and hence it does not keep the tooth whitening effect long. With an amount more than 10 wt %, it separates out excessively on the enamel surface as soon as the gel composition is applied to teeth, and hence it prevents the tooth whitening ingredient from penetrating into the enamel, without fully producing the tooth whitening effect.

According to the present invention, the nonaqueous gel composition for tooth whitening may contain, for its maximum utility, in addition to the above-mentioned tooth whitening ingredient and whitening effect endurance improver, a gelling agent that facilitates its application and adhesion to teeth and prevents it from being diluted by saliva and from dissolving in the mouth. Selection of the gelling agent depends on the form of the gel composition.

The gelling agent used in the present invention is not specifically restricted; it includes polyacrylic acid (homopolymer of acrylic acid), carboxyvinyl polymer, hydroxypropyl cellulose, carboxymethyl cellulose, and salts thereof. Preferable among them are hydroxypropyl cellulose, carboxymethyl cellulose, and salts thereof. They may be used alone or in combination with one another.

The polyacrylic acid used in the present invention is not specifically restricted; however, the one which meets one of the following requirements A to C for viscosity at 25° C. (particularly requirements A and B) is desirable.
Requirement A: The sample should give a 10% aqueous solution having a viscosity of 50,000 to 110,000 mPa·s measured with a BH-type viscometer (rotor No. 7, 20 rpm, 90 sec), from Toki Sangyo Co., Ltd. An example of commercial products meeting this requirement is Jurimer AC-10SH having a viscosity of 70,000 to 110,000 mPa·s, from Nihonjunyaku Co., Ltd.
Requirement B: The sample should give a 20% aqueous solution having a viscosity of 10,000 to 50,000 mPa·s measured with a BM-type viscometer (rotor No. 4, 12 rpm, 90 sec), from Toki Sangyo Co., Ltd. An example of commercial products meeting this requirement is Jurimer AC-10H having a viscosity of 20,000 to 40,000 mPa·s, from Nihonjunyaku Co., Ltd.
Requirement C: The sample should give a 40% aqueous solution having a viscosity of 1000 to 4000 mPa·s measured with a BM-type viscometer (rotor No. 3, 30 rpm, 90 sec), from Toki Sangyo Co., Ltd. An example of commercial products meeting this requirement is Jurimer AC-10L having a viscosity of 1000 to 2000 mPa·s, from Nihonjunyaku Co., Ltd.

The carboxyvinyl polymer used in the present invention is not specifically restricted; however, it should preferably be one which gives a 0.2% aqueous solution (neutralized with sodium hydroxide) having a viscosity of 2000 to 10,000 mPa·s (at 25° C.) measured with a BM-type viscometer (rotor No. 3 to No. 7, 20 rpm, 2 min), from Toki Sangyo Co., Ltd. It includes such commercial products as Carbopol 934, 940, and 941 (from B. F. Goodrich), Hiviswako 103, 104, 105, 204, and 304 (from Wako Pure Chemical Industries,Ltd.), and Junron PW110 and 111 (from Nihonjunyaku).

The carboxyvinyl polymer may optionally be neutralized with a basic substance to adjust its gel-like property (which depends on the degree of neutralization). The substance to be used for neutralization includes inorganic bases (such as sodium hydroxide and potassium hydroxide), triethanolamine, diethanolamine, monoethanolamine, and isopropanolamine.

The hydroxypropyl cellulose used in the present invention is not specifically restricted; however, it should preferably be one which gives a 2% aqueous solution having a viscosity of 6 to 4000 mPa·s, more desirably 150 to 4000 mPa·s, (at 20° C.) measured with a cylindrical rotary viscometer of TVB-20L type (rotor No. L, M2, or M4, 60 rpm, 4 min), from Tokimec Co., Ltd. Its commercial products meeting the first requirement are HPC-L, HPC-M, and HPC-H, and those meeting the second requirement are HPC-M and HPC-H (all from Nippon Soda Co., Ltd.).

The carboxymethyl cellulose and salt thereof used in the present invention are not specifically restricted; however, they should preferably be one which gives a 1 wt % solution having a viscosity of 10 to 4000 mPa·s, more desirably 10 to 300 mPa·s, (at 25° C.) measured with a BL-type viscometer (rotor No. 1 to 4, 60 rpm, 2 min), from Toki Sangyo Co., Ltd.) and which has a degree of etherification of 0.6 to 1.5, more desirably 0.6 to 1.0, measured according to the nonaqueous titration method provided in the Japanese standards of cosmetics ingredients (annotation to the first edition). Its commercial products meeting the first requirement are 1120, 1150, 1190, 1220, 1270, 1310, and 1390, and those meeting the second requirements are 1120, 1150, 1220, 1270 (all from Daicel Chemical Industries, Ltd.).

The amount of the gelling agent should preferably be 0.1 to 15 wt %, particularly 0.5 to 12 wt %, so that it helps the tooth whitening nonaqueous gel composition to stick and fix to teeth particularly in the case where the gel composition is used in combination with tape, sheet, film, or tooth cover. With an amount less than 0.1 wt %, it will not produce its sticking effect, it will be poor in the whitening effect which is produced by entrance of saliva, or it dissolves in the mouth to cause an unpleasant feeling. With an amount more than 15 wt %, it does not dissolve completely and uniformly but prevents the tooth whitening ingredient from dissolution.

According to the present invention, the nonaqueous gel composition for tooth whitening should have a value of hardness measured (at 25° C.) in the following manner. The sample is filled into a cylindrical container (36 mm in diameter and 18 mm deep) up to its top. A plunger (20 mm in diameter) is pushed into the sample at a rate of 20 mm/min and the maximum stress required for the plunger to reach a depth of 10 mm is measured by using a rheometer (CR-200D, from Sun Scientific. Co., Ltd.). The value of hardness thus measured is not specifically restricted; however, it should preferably be 0.001 to 10 kg, more desirably 0.005 to 1 kg, according to the tool to be used. With a value of hardness smaller than 0.001 kg, the nonaqueous gel composition for tooth whitening does not stick and fix to teeth even though it is used in combination with any of tape, sheet, film, and tooth cover, or it dissolves in the mouth, producing an unpleasant feeling, even when it is used in combination with a mouthpiece. With a value of hardness larger than 10 kg, the nonaqueous gel composition for tooth whitening does permit the tooth whitening ingredient to dissolve and hence does not produce the tooth whitening effect.

The nonaqueous gel composition for tooth whitening of the present invention may optionally be incorporated with additional ingredients as listed below.

Surfactants (for dissolution, emulsification, and dispersion), such as anionic surfactants, nonionic surfactants, and amphoteric surfactants. One or more species of surfactants may be used in an amount of 0.05 to 5 wt %, preferably 0.05 to 3 wt %, of the total composition.

Examples of anionic surfactants include

sodium alkyl sulfate such as sodium lauryl sulfate, sodium myristyl sulfate, and sodium cetyl sulfate; sodium N-acylglutamate such as sodium N-lauroyl glutamate and sodium N-palmitoyl glutamate; sodium N-acylsarcosine such as sodium N-lauroyl sarcosine and sodium N-myristoyl sarcosine; sodium N-methyl-N-acyltaurin such as sodium N-lauroyl-methyl-taurin and sodium N-myristoyl-methyl taurin; sodium N-methyl-N-acylalanine, sodium laurylbenzene sulfonate, sodium hydrogenated coconut fatty acid monoglyceride monosulfate, sodium laurylsulfoacetate, sodium α-olefinsulfonate, sodium lauryl POE sulfate, sodium lauryl POE acetate, sodium lauryl POE phosphate, and sodium stearyl POE phosphate.

Examples of nonionic surfactants include glycerin fatty acid ester such as monoglyceryl stearate and decaglycerol laurate, sugar fatty acid ester such as sucrose fatty acid ester, maltose fatty acid ester, and lactose fatty acid ester, sugar alcohol fatty acid ester such as maltitol fatty acid ester and lactitol fatty acid ester, polyoxyethylene sorbitan fatty acid ester such as polyoxyethylene sorbitan monolaurate and polyoxyethylene sorbitan monostearate, polyoxyethylene fatty acid ester such as polyoxyethylene hardened caster oil, fatty acid ethanolamide such as mono- or diethanolamide myristate, sorbitan fatty acid ester, polyoxyethylene higher alcohol ether, polyoxyethylene polyoxypropylene copolymer, and polyoxyethylene polyoxypropylene fatty acid ester.

Examples of amphoteric surfactants include alkyldimethylaminoacetic acid betaine such as lauryl dimethylaminoacetic acid betaine, N-alkyldiaminoethylglycine such as N-laurylaminoethylglycine and N-myristyldiaminoethylglycine, N-alkyl-N-carboxymethylammonium betaine, and sodium 2-alkyl-1-hydroxyethylimidazoline betaine.

The nonaqueous gel composition for tooth whitening of the present invention may be further incorporated with one or more adjuvants exemplified below. Enzymes such as dextrinase, mutanase, lysozyme, amylase, protease, lytic enzyme, and superoxide dismutase, water-soluble polyphosphates (such as tetrapotassium pyrophosphate, sodium tripolyphosphate, and sodium metaphosphate), allantoin, dihydrocholestanol, glycyrrhizic acid, glycyrrhetic acid, ε-aminocaproic acid, tranexamic acid, bisabolol, isopropylmethyl phenol, sodium chloride, triclosan, chlorhexidine salt, cetylpyridinium chloride, benzethonium chloride, benzalkonium chloride, ascorbic acid and salt thereof, tocopherol, and crude drug extracts such as scutellaria root, phellodendron bark, rosemary, clove, and thyme.

The nonaqueous gel composition for tooth whitening of the present invention may be incorporated further with a flavor such as anethole, carvone, peppermint, and spearmint, preservative such as benzoic acid and sodium salt thereof and paraben, dye or colorant such as Red No. 3, Red No. 104, Yellow No. 4, Blue No. 1, Green No. 3, mica titanium, and red iron oxide, and sweetener such as saccharin sodium, stevioside, glycyrrizin, and aspartame.

The nonaqueous gel composition for tooth whitening is not specifically restricted in pH so long as it has a pH value harmless to the mouth and human body. When it is diluted ten times with purified water, the resulting water phase should have a pH value of 4.0 to 10.0, preferably 5.5 to 9.0. If the thus measured pH value is lower than 4.0 or higher than 10.0, it will suffer deashing or have poor taste, respectively. Adequate pH adjustment may be achieved by incorporation with any of acetic acid, hydrochloric acid, sulfuric acid, nitric acid, citric acid, phosphoric acid, sodium hydroxide, potassium hydroxide, sodium acetate, sodium carbonate, sodium citrate, sodium hydrogen citrate, sodium phosphate, and sodium hydrogen phosphate.

The present inventors previously disclosed in their patent application (PCT Patent Publication No. WO03/030851) that the liquid component in the tooth whitening composition should have an Abbe refractive index of 1.35 to 1.50 (measured with sodium D-line at 20° C.) and that the ratio (by weight) of the whitening ingredient to water should be larger than 30/70. Unlike the previous one, the tooth whitening nonaqueous gel composition of the present invention is substantially free of water and becomes poor in tooth whitening effect when diluted with saliva.

The nonaqueous gel composition for tooth whitening of the present invention is substantially free of water; however, there will be an instance in which it contains a small amount of water resulting from pH adjusting agent, moisture absorption by raw materials, and moisture absorption by the gel composition itself. Consequently, the term “substantially free of water” means that the water content is less than 5 wt %, preferably less than 3 wt %, of the total amount of the gel composition.

The nonaqueous gel composition for tooth whitening of the present invention may be prepared in the form of liquid, paste, gel, or foam. For enhanced effect, it may be used in combination with a tool that permits it to remain on and fix to the teeth.

As mentioned above, the nonaqueous gel composition for tooth whitening of the present invention should preferably be applied to teeth in concert with a special tool which retains and keeps it in position in contact with teeth. Such a tool not only helps the gel composition to fix to the teeth but also prevents the gel composition from dissolving in the mouth and removal from the teeth. Dissolution will give an unpleasant feeling to the gum, tongue, and oral mucous membrane and induce salivation. Removal is caused by entrance of saliva and bite, chewing, and other physical stimuli. The tool is not specifically restricted in its material and shape so long as it achieves the above-mentioned object. It may be formed from a water-insoluble material and it may be in the form of tape, sheet, film, mouth tray, mouth piece, sponge, impression material, pack material, tooth cover conforming to the dentition, or chewing brush conforming to the dentition which has many projections on its surface in contact with teeth.

The above-mentioned tool should preferably have a thickness of 0.01 to 5 mm so that it does not give feeling of physical disorder when it is placed in the mouth. The thickness of tape, sheet, or film should preferably be 0.01 to 2 mm.

The above-mentioned tool should preferably be formed from a material which gives a pleasant feeling in the mouth and prevents excess salivation during use, thereby permitting the gel composition to stay in the mouth for a long period of time. Examples of the material include polyethylene, foamed polyethylene, polypropylene, foamed polypropylene, polyester, polyurethane, rayon, pulp, cotton, silk, paper, and metal foil. They may be used alone or in combination with one another. Preferred examples are polyethylene, foamed polyethylene, polypropylene, foamed polypropylene, polyester, polyurethane, and rayon. The tool may be formed from two kinds of materials. In other words, that side of the tool which comes into contact with the dental mucosa and tongue should preferably be formed from a hydrophilic water-absorbing material, such as cloth or non-woven fabric of rayon, pulp, cotton, silk, or paper. Such a material absorbs saliva, thereby giving a pleasant feeling. That side of the tool which holds the gel composition should preferably be formed from a water-impermeable film of polyethylene, polypropylene, polyester, or polyurethane. Such a film prevents the gel composition from sticking to or infiltrating into the tool for application.

On the other hand, it is desirable to form the tray, mouth piece, and chewing brush from silicone rubber, natural rubber, vinyl acetate resin, acrylic resin, or ethylene-vinyl acetate resin, which easily deforms to fit to the user's dentition and tooth shape. The resulting tool firmly sticks to teeth and makes the gel composition to produce its effect for a long period of time.

In the case where the nonaqueous gel composition for tooth whitening has a low viscosity (and hence it does not firmly stick to teeth), it is possible to cover the inside of the tooth cover or tray with a sponge (or the like made of water-absorbing material such as rayon, cotton, and pulp) impregnated with the gel composition. The user chews the tooth cover or tray continuously so that the gel composition in a proper amount is applied to the teeth.

The frequency and duration of application should be properly selected. Usually, once to six times a day (particularly once to thrice a day) and 1 to 120 minutes (particularly 1 to 60 minutes) for one dosage. Application while sleeping is also possible.

### EXAMPLES

The invention will be described in more detail with reference to the following examples and comparative examples, which are not intended to restrict the scope thereof. Incidentally, “%” in each example refers to “% by weight”, further tooth whitening ingredient used in the following examples all have a relative permittivity of 17.0 to 43.0 (at 25° C.) and a vapor pressure of 0 to 7000 mPa·s (at 25° C.). t-But stands for tert-butyl group.

<Measurement of Relative Permittivity>

The sample (100 mL) was tested for relative permittivity at 25° C. at a frequency of 100 kHz and a voltage of 1 V by using an LCR meter (HP4284A, with HP16452A electrode for liquid, from Hewlett Packard).

<Measurement of Vapor Pressure>

The sample (80 mL in a cell) was tested for vapor pressure at (25° C.) by using a vapor-liquid equilibrium measuring apparatus (VLE autolabo, from TOYO KOATSU Co., Ltd.).

<Evaluation of Solubility of the Ingredient B—Whitening Effect Endurance Improver>

A sample (0.1 g) of the ingredient (B) is added to 100 g of the tooth whitening ingredient (A), and the resulting mixture is stirred at 37° C. for 16 hours and allowed to stand at 37° C. for 24 hours. The mixture is visually examined for undissolved residues of the ingredient (B).

Solubility of the ingredient (B) in an aqueous solution of 1 mmol/L calcium chloride was determined in the same way as above. The results are shown in Tables 1 and 2.

Criterion for Solubility:
◯: solubility more than 0.1 g (no undissolved residues)
X: solubility less than 0.1 g (with undissolved residues)

Criterion for Overall Rating:
◯: More than 0.1 g is soluble in 100 g of ingredient (A) and less than 0.1 g is soluble in 100 g of aqueous solution of 1 mmol/L calcium chloride.

X: Other than specified above.

TABLE 1

| | | Whitening effect endurance improver (B) | | | | | | | | | |
|---|---|---|---|---|---|---|---|---|---|---|---|
| | | | | | | | | | | | Lauric acid |
| | | Isostearic | 12-hydroxy | Oleic | Linoleic | Palmitoleic | Linolenic | Myristic | Erucic | | (Comparative |
| | | acid | stearic acid | acid | acid | acid | acid | acid | acid | Shellac | Example) |
| Tooth | Ethylene glycol | | | | | | | | | ◯ | |
| whitening | Propylene glycol | | | | | ◯ | ◯ | | | ◯ | |
| ingredient | 1,3-butylene glycol | ◯ | ◯ | | | ◯ | | | | ◯ | ◯ |
| (A) | Isopropanol | | | | | | | | | | |
| | Diethylene glycol | ◯ | | | | | | | | | |
| | Dipropylene glycol | ◯ | | | | | | | | ◯ | |
| | Glycerin/1,3-butylene | ◯ | | | | | | | | | |
| | glycol = 7/3 | | | | | | | | | | |
| | PEG#300 | ◯ | | ◯ | ◯ | ◯ | ◯ | ◯ | ◯ | ◯ | |
| | (Av. MW 280-320) | | | | | | | | | | |
| | PEG#400 | ◯ | | | | | | | | ◯ | |
| | (Av. MW 380-420) | | | | | | | | | | |
| | PEG#600 | | | | | | ◯ | | | ◯ | |
| | (Av. MW 570-630) | | | | | | | | | | |

| | | | | | | | | | | |
|---|---|---|---|---|---|---|---|---|---|---|
| Aqueous solution of | X | X | X | X | X | X | X | X | X | ◯ |
| 1 mmol/L calcium chloride | | | | | | | | | | |
| Overall rating | ◯ | ◯ | ◯ | ◯ | ◯ | ◯ | ◯ | ◯ | ◯ | X |

TABLE 2

| | | Whitening effect endurance improver (B) | | | |
|---|---|---|---|---|---|
| | | t-butyl acrylate/ethyl | Acrylic acid/acrylamide/ | Acrylic acid/acrylamide/ | Octylacrylamide/acrylate ester |
| | | acrylate copolymer | ethyl acrylate copolymer | ethyl acrylate copolymer | copolymer |
| | | (Luvimer 100P, from | (Ultrahold 8, | (Ultrahold strong, | (Amphomer V-42, |
| | | BASF) | from BASF) | from BASF) | from Nippon NSC) |
| Tooth whitening | Ethylene glycol | | | | |
| ingredient (A) | Propylene glycol | | | ◯ | ◯ |
| | 1,3-butylene glycol | ◯ | | | |
| | Isopropanol | | | ◯ | |
| | Diethylene glycol | | | | |
| | Dipropylene glycol | | | | ◯ |
| | Glycerin/1,3-butylene | | | | |
| | glycol = 7/3 | | | | |
| | PEG#300 | ◯ | ◯ | ◯ | ◯ |
| | (Av. MW 280-320) | | | | |
| | PEG#400 | | | | |
| | (Av. MW 380-420) | | | | |
| | PEG#600 | ◯ | | | |
| | (Av. MW 570-630) | | | | |

| | | | | |
|---|---|---|---|---|
| Aqueous Solution of | X | X | X | X |
| 1 mmol/L calcium chloride | | | | |
| Overall rating | ◯ | ◯ | ◯ | ◯ |

| | | Whitening effect endurance improver (B) | | | |
|---|---|---|---|---|---|
| | | | | Methyl methacrylate/ | |
| | | Methyl methacrylate/ | | ethyl acrylate/ | |
| | | methacrylic | Methyl acrylate/methacrylic | trimethyl ammonium ethyl | |
| | | acid copolymer | acid copolymer | methacrylate copolymer | Zinc chloride |
| | | (Eudragit S100, | (Eudragit L100, | (Eudragit RSPO, | (Comparative |
| | | from Rohm GmbH) | from Rohm GmbH) | from Rohm GmbH) | Example) |
| Tooth whitening | Ethylene glycol | | | | |
| ingredient (A) | Propylene glycol | | | | |
| | 1,3-butylene glycol | | ◯ | ◯ | ◯ |
| | Isopropanol | ◯ | | | |
| | Diethylene glycol | | | | |
| | Dipropylene glycol | | ◯ | | |
| | Glycerin/1,3-butylene | | | ◯ | |
| | glycol = 7/3 | | | | |
| | PEG#300 | ◯ | ◯ | ◯ | |
| | (Av. MW 280-320) | | | | |
| | PEG#400 | | | ◯ | |
| | (Av. MW 380-420) | | | | |
| | PEG#600 | | | | |
| | (Av. MW 570-630) | | | | |

| | | | | |
|---|---|---|---|---|
| Aqueous Solution of | X | X | X | ◯ |
| 1 mmol/L calcium chloride | | | | |
| Overall rating | ◯ | ◯ | ◯ | X |

### Examples 1 to 11 and Comparative Examples 1 to 6

<Preparation of Nonaqueous Gel Composition for Tooth Whitening>

All raw materials (except flavor) were weighed and mixed at about 80° C. under a reduced pressure of 8.0 kPa. After standing for a prescribed period of time, a flavor was added at 20 to 40° C. and mixed under a reduced pressure to give a desired product. Sodium hydroxide or potassium hydroxide was added for pH adjustment so that the product gives an aqueous solution (diluted 10 times (w/w) with purified water) that has a pH of 4.0 to 10.0 at 25° C.

<Evaluation of Endurance of Tooth Whitening Effect>

An extracted human tooth which had previously been examined for color difference (L*0, a*0, b*0) was covered with a polyurethane film (20 mm by 20 mm, 50 μm thick) coated with 1.0 g of the gel composition having a composition shown in Tables 3 and 4. (The polyurethane film is DUS2124-CDB from Sheedom Co., Ltd.) The tooth specimen was allowed to stand in a thermostat at 37° C. for 1 hour. The polyurethane film was removed and the tooth specimen was cleaned of gel by wiping with tissue paper. After slight water washing, the tooth specimen was immersed in artificial saliva for 3 hours. The tooth specimen was examined again for color difference (L*1, a*1, b*1), and the value of ΔE was calculated from the following formula.
ΔE=((L*1−L*0)²+(a*1−a*0)²+(b*1−b*0)²)^{1/2}
The value of ΔE is regarded as the measure of endurance of the tooth whitening effect. The color difference was measure by using a spectrophotometer (CM-2022, from Minolta). The results are shown in Tables 3 and 4.

Criterion for Rating:

⊚: ΔE≧4.0

◯: 3.0≦ΔE<4.0

X: ΔE<3.0

TABLE 3

| | | Vapor | | | | | | | | | | | |
|---|---|---|---|---|---|---|---|---|---|---|---|---|---|
| | Relative | pressure | | | | | | | | | | | |
| Ingredient | Permittivity | kPa | Example | | | | | | | | | | |
| (%) | (at 25° C.) | (at 25° C.) | 1 | 2 | 3 | 4 | 5 | 6 | 7 | 8 | 9 | 10 | 11 |
| Dipropylene glycol | 26.2 | 0 | 92 | | | | | | | | | | |
| 1,3-dibutylene glycol | 33.5 | 108 | | 92 | 35 | 62 | 92.9 | 92 | 88 | 55 | 92 | 92 | 92 |
| Glycerin | 42.5 | 3 | | | 60 | | | | | | | | |
| Ethanol | 24.6 | 7948 | | | | 30 | | | | 18 | | | |
| Water | 78.3 | 3165 | | | | | | | | | | | |

| | | | | | | | | | | | |
|---|---|---|---|---|---|---|---|---|---|---|---|
| Isostearic acid | 1 | 1 | 1 | 1 | 0.1 | | | | | | |
| 12-hydorxystearic acid | | | | | | 1 | | | | | |
| Shellac | | | | | | | 5 | 5 | | | |
| t-Bu acrylate/ethyl acrylate/ | | | | | | | | | 1 | | |
| methacrylic acid copolymer | | | | | | | | | | | |
| (Luvimer 100P, from BASF) | | | | | | | | | | | |
| Methyl methacrylate/ethyl acrylate/ | | | | | | | | | | 1 | |
| trimethyl-ammonium ethyl methacrylate | | | | | | | | | | | |
| copolymer (Eudragit RSPO, from Rohm) | | | | | | | | | | | |
| Methyl acrylate/methacrylic acid | | | | | | | | | | | 1 |
| copolymer | | | | | | | | | | | |
| (Eudragit L100, from Rohm) | | | | | | | | | | | |
| Hydroxypropyl cellulose | 6 | 6 | | 6 | 6 | 6 | 6 | 12 | 6 | 6 | 6 |
| (HPC-H, from Nippon Soda Co., Ltd.) | | | | | | | | | | | |
| Sodium carboxymethyl cellulose | | | 3 | | | | | | | | |
| (1240, from Daicel Chemical | | | | | | | | | | | |
| Industries, Ltd.) | | | | | | | | | | | |
| Thickening silica | | | | | | | | 6 | | | |
| (Tokuseal, from Tokuyama Corp.) | | | | | | | | | | | |
| Sodium laurate | | | | | | | | 2 | | | |
| Citric acid | | | | | | | | 0.3 | | | |
| Sodium citrate | | | | | | | | 0.7 | | | |
| Flavor | 1 | 1 | 1 | 1 | 1 | 1 | 1 | 1 | 1 | 1 | 1 |
| Total | 100 | 100 | 100 | 100 | 100 | 100 | 100 | 100 | 100 | 100 | 100 |
| Endurance of | ◯ | ⊚ | ⊚ | ◯ | ⊚ | ◯ | ⊚ | ◯ | ⊚ | ⊚ | ◯ |
| tooth whitening effect ΔE | | | | | | | | | | | |

TABLE 4

| | | Vapor | | | | | | |
|---|---|---|---|---|---|---|---|---|
| | Relative | pressure | | | | | | |
| Ingredient | Permittivity | kPa | Comparative Example | | | | | |
| (%) | (at 25° C.) | (at 25° C.) | 1 | 2 | 3 | 4 | 5 | 6 |
| 1,3-dibutylene glycol | 33.5 | 108 | 93 | 98 | 92 | | 82 | 92 |
| Ethanol | 24.6 | 7948 | | | | 92 | | |
| Water | 78.3 | 3165 | | | | | 10 | |

| | | | | | | |
|---|---|---|---|---|---|---|
| Isostearic acid | | 1 | | 1 | | |
| Lauric acid | | | 1 | | | |
| t-Bu acrylate/ethyl acrylate/ | | | | | 1 | |
| methacrylic acid copolymer | | | | | | |
| (Luvimer 100P, from BASF) | | | | | | |
| Hydroxypropyl cellulose | 6 | | 6 | 6 | 6 | 6 |
| (HPC-H, from Nippon Soda Co., Ltd.) | | | | | | |
| Zinc chloride | | | | | | 1 |
| Flavor | 1 | 1 | 1 | 1 | 1 | 1 |
| Total | 100 | 100 | 100 | 100 | 100 | 100 |
| Endurance of | X | X | X | X | X | X |
| tooth whitening effect ΔE | | | | | | |

It is concluded from the above-mentioned experiment that the reversible tooth whitening effect is maintained for a prolonged period of time when the nonaqueous gel composition is incorporated with a tooth whitening ingredient which has a relative permittivity of 17.0 to 43.0 (at 25° C.) and a vapor pressure of 0 to 7000 kPa (at 25° C.) and which separates out upon contact with an aqueous solution of calcium chloride, a particular example of the ingredient being a C₁₄₋₂₂ higher fatty acid or an acrylic acid copolymer.

### Example 12

The nonaqueous gel composition for tooth whitening 1 specified below was prepared in the same way as in Examples 1 to 11 and Comparative Examples 1 to 6. The upper first front tooth was previously examined for color difference. The sample (2.0 g) was applied to upper six teeth (from the third left to the third right). The inside of the upper lip facing the six teeth was covered with a foamed polyethylene sheet (15 mm by 60 mm, 800 μm thick, Volara XL-IF08008, from Sekisui Chemical Co., Ltd.). One hour later, the sheet was removed and the teeth were cleaned of the gel composition by wiping with tissue paper. The test subject was allowed to stay quiet for 3 hours. The upper first front tooth was examined again for color difference, and the endurance of the tooth whitening effect was evaluated in the same way as mentioned above. The rating was ◯.

(Nonaqueous gel composition for tooth whitening 1)

| | |
|---|---|
| Polyethylene glycol #300 | 93.0 |
| (Av. MW = 280 to 320, relative permittivity = 32.0, | |
| vapor pressure = 0 kPa) | |
| Linoleic acid | 1.0 |
| Hydroxypropyl cellulose | 6.0 |
| (HPC-H, from Nippon Soda Co., Ltd.) | |
| Total | 100.0% |

### Example 13

The nonaqueous gel composition for tooth whitening 2 specified below was prepared in the same way as in Examples 1 to 11 and Comparative Examples 1 to 6. The sample (2.0 g) was applied to upper six teeth (from the third left to the third right). The inside of the upper lip facing the six teeth was covered with a polyurethane film (15 mm by 60 mm, 50 μm thick, DUS2124-CDB, from Sheedom). The endurance of the tooth whitening effect was evaluated in the same way as in Example 12. The rating was ◯.

(Nonaqueous gel composition for tooth whitening 2)

| | |
|---|---|
| Propylene glycol | 50.0 |
| (relative permittivity = 32.0, vapor pressure = 1 kPa) | |
| Dipropylene glycol | 36.9 |
| (relative permittivity = 26.2, vapor pressure = 0 kPa) | |
| Shellac (Dry clear lac, from Nippon Shellac) | 7.0 |
| Acrylate octylacrylamide/acrylate ester copolymer | 1.0 |
| (Amphomer V-42, from Nippon NSC) | |
| Hydroxypropyl cellulose | 5.0 |
| (HPC-H, from Nippon Soda Co., Ltd.) | |
| Saccharin | 0.1 |
| Total | 100.0% |

### Example 14

The nonaqueous gel composition for tooth whitening 3 specified below was prepared in the same way as in Examples 1 to 11 and Comparative Examples 1 to 6. A polyethylene film of the shape of single tooth which is coated with 0.2 g of the sample was applied to the upper first front tooth. (The polyethylene sheet is a flexible polyethylene sheet, 10 mm by 15 mm, 25 μm thick, from Toray). The endurance of the tooth whitening effect was evaluated in the same way as in Example 12. The rating was ⊚.

(Nonaqueous gel composition for tooth whitening 3)

| | |
|---|---|
| Polyethylene glycol #400 | 67.29 |
| (Av. MW = 380 to 420, relative permittivity = 32.0, | |
| vapor pressure = 0 kPa) | |
| Glycerin | 25.0 |
| (relative permittivity = 42.5, vapor pressure = 3 kPa) | |
| Shellac (Dry clear lac, from Nippon Shellac) | 1.0 |
| Hydroxypropyl cellulose | 5.0 |
| (HPC-H, from Nippon Soda Co., Ltd.) | |
| Sodium carboxymethylcellulose | 0.5 |
| (1290, from Daicel Chemical Industries, Ltd.) | |
| Sodium hydroxide | 0.01 |
| Flavor | 1.2 |
| Total | 100.0% |

### Example 15

The nonaqueous gel composition for tooth whitening 4 specified below was prepared in the same way as in Examples 1 to 11 and Comparative Examples 1 to 6. The sample (1.0 g) was applied by using a brush to upper six teeth (from the third left to the third right). A flexible polypropylene film (15 mm by 60 mm, 100 μm thick, from Toray) was attached to the teeth. The endurance of the tooth whitening effect was evaluated in the same way as in Example 12. The rating was ◯.

(Nonaqueous gel composition for tooth whitening 4)

| | |
|---|---|
| Diethylene glycol | 90.85 |
| (relative permittivity = 33.0, vapor pressure = 11 kPa) | |
| Menthol | 1.0 |
| Polyacrylic acid | 8.0 |
| (Ac-10H, from Nihonjunyaku Co., Ltd) | |
| Sodium hydroxide | 0.05 |
| Isostearic acid | 0.1 |
| Total | 100.0% |

### Example 16

The nonaqueous gel composition for tooth whitening 5 specified below was prepared in the same way as in Examples 1 to 11 and Comparative Examples 1 to 6. The sample (1.0 g) was applied to a mouthpiece, which was subsequently placed on teeth. (The mouthpiece was prepared from “Nightguard” of ethylene-vinyl acetate copolymer resin (from Dental Concept) which each test subject chewed after softening by immersion in hot water to make it fit to him. The endurance of the tooth whitening effect was evaluated in the same way as in Example 12. The rating was ◯.

(Nonaqueous gel composition for tooth whitening 5)

| | |
|---|---|
| Isopropanol | 70.0 |
| (relative permittivity = 19.9, vapor pressure = 5819 kPa) | |
| Ethanol | 24.8 |
| (relative permittivity = 24.6, vapor pressure = 7948 kPa) | |
| Methyl methacrylate/methacrylic acid copolymer | 0.1 |
| (Eudragit S100, from Rohm GmbH) | |
| Carboxyvinyl polymer (CVP980, from BF Goodrich) | 5.0 |
| Sodium hydroxide | 0.1 |
| Total | 100.0% |

### Example 17

The nonaqueous gel composition for tooth whitening 6 specified below was prepared in the same way as in Examples 1 to 11 and Comparative Examples 1 to 6. The sample (0.5 g) was impregnated into a sheet of rayon nonwoven fabric, 10 mm by 40 mm (“Pilos” spunrace, rayon 100%, basis weight 40 g/m², from Omi Kenshi). The impregnated sheet was applied to upper front teeth. The endurance of the tooth whitening effect was evaluated in the same way as in Example 12. The rating was ◯.

(Nonaqueous gel composition for tooth whitening 6)

| | |
|---|---|
| Propylene glycol | 74.99 |
| (relative permittivity = 32.0, vapor pressure = 1 kPa) | |
| 1,3-butylene glycol | 21.0 |
| (relative permittivity = 33.5, vapor pressure = 108 kPa) | |
| Palmitoleic acid | 3.0 |
| Caboxyvinyl polymer | 1.0 |
| (Junron PW110, from Nihonjunyaku Co., Ltd) | |
| Potassium hydroxide | 0.01 |
| Total | 100.0% |

### Example 18

The nonaqueous gel composition for tooth whitening 7 specified below was prepared in the same way as in Examples 1 to 11 and Comparative Examples 1 to 6. The sample (2 g) was applied to the nonwoven fabric side of the laminate sheet, 30 mm by 60 mm, 0.5 mm thick, composed of polyethylene film and polypropylene/rayon nonwoven fabric. The former is low-density polyethylene film, 10 μm thick, grade No. 212, from Tosoh, and the latter is nonwoven fabric of polypropylene/rayon=3/7, spunrace, basis weight 40 g/m², grade No. 7140-6, from Shinwa Co., Ltd. The laminate sheet was attached to upper front six teeth (from the left third to the right third). The sheet was partly folded back toward the tongue surface. The endurance of the tooth whitening effect was evaluated in the same way as in Example 12. The rating was ◯.

(Nonaqueous gel composition for tooth whitening 7)

| | |
|---|---|
| Polyethylene glycol #600 | 78.99 |
| (Av. MW = 570 to 630, relative permittivity = 33.0, | |
| vapor pressure = 0 kPa) | |
| Propylene glycol | 10.0 |
| (relative permittivity = 32.0, vapor pressure = 1 kPa) | |
| Linolenic acid | 5.0 |
| Hydroxypropyl cellulose | 6.0 |
| (HPC-H, from Nippon Soda Co., Ltd.) | |
| 2,6-di-t-butylhydroxytoluene | 0.01 |
| Total | 100.0% |

### Example 19

The nonaqueous gel composition for tooth whitening 8 specified below was prepared in the same way as in Examples 1 to 11 and Comparative Examples 1 to 6. The sample (2.0 g) was applied to a polyester tape 10 mm by 40 mm, 0.05 mm thick (Grade S, from Teijin Dupont Film). The polyester tape was attached to upper front teeth, with the sample facing inside. The endurance of the tooth whitening effect was evaluated in the same way as in Example 12. The rating was ◯.

(Nonaqueous gel composition for tooth whitening 8)

| | |
|---|---|
| Dipropylene glycol | 87.8 |
| (relative permittivity = 26.2, vapor pressure = 0 kPa) | |
| Methyl acrylate/methacrylic acid copolymer | 1.0 |
| (Eudragit L100, from Rohm GmbH) | |
| Hydroxypropyl cellulose | 8.0 |
| (HPC-H, from Nippon Soda Co., Ltd.) | |
| Sodium lauryl sulfate | 2.0 |
| Flavor | 1.0 |
| Saccharin sodium | 0.2 |
| Total | 100.0% |

### Example 20

The nonaqueous gel composition for tooth whitening 9 specified below was prepared in the same way as in Examples 1 to 11 and Comparative Examples 1 to 6. The sample (0.5 g) was applied to the inside of the tooth cover prepared in the following manner and the tooth cover was fitted onto teeth. First, a three-layer laminate sheet was formed by fusion-bonding two sheets of nonwoven fabric, with a polyethylene film interposed between them. One nonwoven fabric is embossed polypropylene nonwoven fabric (polypropylene 100%, spunbond, basis weight 40 g/m², Idemitsu RN2040, from Idemitsu Kosan Co., Ltd.), and the other nonwoven fabric is a rayon/polypropylene nonwoven fabric (rayon/polypropylene=70/30, spunrace, basis weight 40 g/m², Grade 7140-6, from Shinwa Co., Ltd.). The laminate sheet was molded into a tooth cover (with heating under pressure) by using a gypsum mold, such that the polypropylene nonwoven fabric faces inside. The endurance of the tooth whitening effect was evaluated in the same way as in Example 12. The rating was ⊚.

(Nonaqueous gel composition for tooth whitening 9)

| | |
|---|---|
| Ethylene glycol | 90.85 |
| (relative permittivity = 37.7, vapor pressure = 59 kPa) | |
| Shellac | 1.0 |
| Hydroxypropyl cellulose | 6.0 |
| (HPC-H, from Nippon Soda Co., Ltd.) | |
| Sodium tripolyphosphate | 1.0 |
| Cetylpyridinium chloride | 0.05 |
| Flavor | 1.0 |
| Saccharin sodium | 0.1 |
| Total | 100.0% |

### Example 21

The nonaqueous gel composition for tooth whitening 10 specified below was prepared in the same way as in Examples 1 to 11 and Comparative Examples 1 to 6. The sample (0.2 g) was impregnated into a sponge sheet (10 mm by 40 mm, 1 mm thick, Soft Foam 271S, from Kurabo Industries Ltd.), which was subsequently attached to upper front teeth. The endurance of the tooth whitening effect was evaluated in the same way as in Example 12. The rating was ⊚.

(Nonaqueous gel composition for tooth whitening 10)

| | |
|---|---|
| Polyethylene glycol #400 | 55.9 |
| (Av. MW = 380 to 420, relative permittivity = 32.0, | |
| vapor pressure = 0 kPa) | |
| Glycerin | 40.0 |
| (relative permittivity = 42.5, vapor pressure = 3 kPa) | |
| Methyl methacrylate/ethyl acrylate/ | 1.0 |
| trimethyl ammonium ethyl methacrylate copolymer | |
| (Eudragit RSPO, from Rohm GmbH) | |
| Hydroxypropyl cellulose | 2.0 |
| (HPC-H, from Nippon Soda Co., Ltd.) | |
| Sodium carboxymethylcellulose | 0.5 |
| (1150, from Daicel Chemical Industries, Ltd.) | |
| Flavor | 0.5 |
| Saccharin sodium | 0.1 |
| Total | 100.0% |

### Example 22

The nonaqueous gel composition for tooth whitening 11 specified below was prepared in the same way as in Examples 1 to 11 and Comparative Examples 1 to 6. The sample was applied to upper front teeth and then the teeth were covered with a water-insoluble acrylic pack material (which forms a coating film). The endurance of the tooth whitening effect was evaluated in the same way as in Example 12. The rating was ◯.

(Nonaqueous gel composition for tooth whitening 11)

| | |
|---|---|
| Polyethylene glycol #300 | 56.26 |
| (Av. MW = 280 to 320, relative permittivity = 32.0, | |
| vapor pressure = 0 kPa) | |
| Ethanol | 34.0 |
| (relative permittivity = 24.6, vapor pressure = 7948 kPa) | |
| Isostearic acid | 2.0 |
| Sodium lauryl sulfate | 2.0 |
| Polyacrylic acid | 4.0 |
| (AQUPEC HV-501 Nippon Fine Chemical) | |
| Hydroxypropyl cellulose | 1.0 |
| (HPC-L, from Nippon Soda Co., Ltd.) | |
| Flavor | 0.5 |
| Sodium hydroxide | 0.04 |
| Saccharin sodium | 0.2 |
| Total | 100.0% |

<Water-insoluble acrylic pack material>

| | |
|---|---|
| Methyl methacrylate/butyl methacrylate/ | 10.0 |
| dimethyl-aminoethyl methacrylate copolymer | |
| (Eudragit EPO, from Rohm Pharm) | |
| Stearic acid | 1.0 |
| Ethanol | 89.0 |
| Total | 100.0% |

### Example 23

The nonaqueous gel composition for tooth whitening 12 specified below was prepared in the same way as in Examples 1 to 11 and Comparative Examples 1 to 6. The sample (0.5 g) was applied to a denture mold prepared as follows, which was subsequently fitted onto teeth. A prescribed amount of alginate impression material was rapidly mixed with water in a cup, and the resulting mixture was transferred to an impression tray. The impression tray holding the mixture was fitted onto teeth and allowed to stand for a predetermined period of time. The endurance of the tooth whitening effect was evaluated in the same way as in Example 12. The rating was ◯.

(Nonaqueous gel composition for tooth whitening 12)

| | |
|---|---|
| Propylene glycol | 43.5 |
| (relative permittivity = 32.0, vapor pressure = 1 kPa) | |
| Isopropyl alcohol | 24.99 |
| (relative permittivity = 19.9, vapor pressure = 5819 kPa) | |
| Ethanol | 24.0 |
| (relative permittivity = 24.6, vapor pressure = 7948 kPa) | |
| Acrylic acid/acrylamide/ethyl acrylate copolymer | 1.0 |
| (Ultrahold strong, from BASF) | |
| Sodium lauryl sulfate | 3.0 |
| Myristic acid diethanolamide | 1.0 |
| Carboxyvinyl polymer (CVP980, from BF Goodrich) | 1.0 |
| Flavor | 1.0 |
| Sodium hydroxide | 0.01 |
| Stevioside | 0.5 |
| Total | 100.0% |

1. A nonaqueous gel composition for tooth whitening comprising (A) a tooth whitening ingredient having a relative permittivity of 17.0 to 43.0 (at 25° C.) and a vapor pressure of 0 to 7000 kPa (at 25° C.), (B) a substance which dissolves in said tooth whitening ingredient and is precipitated by an aqueous solution of calcium chloride, and (C) a gelling agent, said composition being substantially free of water and peroxide.

2. The nonaqueous gel composition for tooth whitening as defined in claim 1, wherein the tooth whitening ingredient (A) is at least one selected from the group consisting of isopropanol, butanol, ethylene glycol, polyethylene glycol (with an average molecular weight of 190 to 630), diethylene glycol, propylene glycol, dipropylene glycol, butylene glycol, and glycerin.

3. The nonaqueous gel composition for tooth whitening as defined in claim 1 or claim 2, wherein the substance (B) which dissolves in the tooth whitening ingredient and is precipitated by an aqueous solution of calcium chloride is at least one selected from the group consisting of C₁₄₋₂₂ higher fatty acids and/or acrylic acid copolymers.

4. The nonaqueous gel composition for tooth whitening as defined in claim 3, wherein the substance (B) is at least one selected from the group consisting of myristic acid,
7-hydroxymyristic acid, jalaric acid,
9,10,16-trihydroxypalmitic acid, palmitoleic acid,
12-hydroxystearic acid, isostearic acid, oleic acid,
linoleic acid, linolenic acid, erucic acid, shellac,
t-Bu acrylate/ethyl acrylate/methacrylic acid copolymer,
methyl acrylate/methacrylic acid copolymer,
methyl methacrylate/methacrylic acid copolymer,
acrylic acid/acrylamide/ethyl acrylate copolymer,
octylacrylamide/acrylate ester copolymer,
methyl methacrylate/ethyl acrylate/methacrylic acid trimethylammonium ethyl chloride copolymer.

5. The nonaqueous gel composition for tooth whitening as defined in claim 1, wherein the gelling agent (C) is at least one selected from the group consisting of polyacrylic acid, carboxyvinyl polymer, hydroxypropyl cellulose, carboxymethyl cellulose, and salts thereof.

6. The nonaqueous gel composition for tooth whitening as defined in claim 1, wherein the tooth whitening ingredient (A) accounts for 50.0 to 99.5% by weight of the total amount of the composition, the substance (B) accounts for 0.1 to 10% by weight of the total amount of the composition, and the gelling agent (C) accounts for 0.1 to 15% by weight of the total amount of the composition.

7. The nonaqueous gel composition for tooth whitening as defined in claim 1, which further comprises a surfactant that accounts for 0.05 to 5% by weight of the total amount of the composition.

8. A tooth whitening set comprising the nonaqueous gel composition for tooth whitening defined in claim 1 and a tool for its application which is detachably fitted to teeth while holding it.

9. The tooth whitening set as defined in claim 8, wherein the tool for application is any of tape, sheet, film, mouth tray, mouth piece, sponge, impression material, pack material, and tooth cover conforming to the definition, which are all made of water-insoluble material. position.
